(19) United States
(12) Patent Application Publication
(10) Pub. No.: US 2025/0241792 A1
(43) Pub. Date: Jul. 31, 2025

(54) PHACOEMULSIFICATION INSTRUMENTS

(71) Applicant: RAICO International LLC, Westmont, IL (US)

(72) Inventor: Ravi Nallakrishnan, Willowbrook, IL (US)

(21) Appl. No.: 19/023,699

(22) Filed: Jan. 16, 2025

Related U.S. Application Data
(60) Provisional application No. 63/625,722, filed on Jan. 26, 2024.

(51) Int. Cl. A61F 9/007

(52) U.S. Cl. CPC A61F 9/00745

(57) ABSTRACT

A phacoemulsification needle includes a needle shaft portion defining a longitudinal axis and having an internal aspiration passage. The needle includes a flaring emulsification tip at a distal end of the needle shaft portion and extending radially outwardly beyond the needle shaft portion. The emulsification tip includes an anti-glare surface treatment. An infusion sleeve for use with a phacoemulsification needle includes a hollow flexible tubular body for receiving the phacoemulsification needle and includes open proximal and distal ends, and an internal passage through which irrigating fluid passes to a surgical site when the infusion sleeve is mounted around the needle. The infusion sleeve includes at least one pair of adjacent, internal ribs within the internal passage positioned to engage the phacoemulsification needle to minimize thermal damage during a surgical procedure.

### PRIORITY

This application claims priority to U.S. provisional application Ser. No. 63/625,722, filed Jan. 26, 2024, the entire contents of which is incorporated herein by reference.

### FIELD OF THE INVENTION

The present invention relates generally to surgical instruments used in ophthalmological surgery and, more particularly, to improved phacoemulsification needles and irrigation fluid sleeves which are particularly suited for use with an associated ultrasonic vibratory surgical handpiece to facilitate efficient operation and use thereof.

### BACKGROUND OF THE INVENTION

Phacoemulsification has come to be a technique of choice for the removal of damaged or diseased lenses from the eye. Commonly, such surgery is called for when a patient develops cataracts, a condition in which a portion of the eye lens becomes hard and opaque. Unless the damaged lens is removed and replaced with a properly selected artificial lens, blindness or severely impaired vision will result.

Phacoemulsification is the use of ultrasonic energy to emulsify the damaged lens and aspirate the resulting lens particles from the eye. One of the most significant advantages of the use of phacoemulsification is that the apparatus itself is small and can fit through a relatively small incision, resulting in less fluid leakage from the eye capsule and shorter patient recovery times. It is desirable to limit the amount of ultrasonic energy used as much as possible in order to minimize the risk of damage to eye tissue. Often, the lens nucleus (the hardest portion of the lens) is chopped or split into smaller pieces prior to or during phacoemulsification. Smaller pieces require less energy to emulsify, and this shortens the time during which ultrasonic energy is actually being created by the phacoemulsification apparatus.

Commonly, an infusion sleeve is mounted around the needle to supply irrigating liquids to the eye in order to maintain positive pressure in the eye as the emulsified lens nucleus and fluids are aspirated through the hollow lens.

It is extremely important to properly infuse liquid during such surgery. Maintaining a sufficient amount of liquid prevents collapse of certain tissues within the eye and attendant injury or damage to delicate eye structures. As an example, endothelial cells can easily be damaged during such collapse and this damage is permanent because these cells do not regenerate. One of the benefits of using as small an incision as possible during such surgery is the minimization of leakage of liquid during and after surgery to help prevent tissue collapse, faster healing time, and decreased post-operative astigmatism.

U.S. Pat. No. 7,601,136, hereby incorporated by reference, discloses a phacoemulsification needle and sleeve assembly.

Many phacoemulsification needles and tips are designed for use with handpieces that vibrate the needle longitudinally at relatively low frequencies. In addition to longitudinal vibration, certain handpieces impart a torsional motion to the needle at an oscillation frequency of about 100 cycles per second. There are also handpieces that provide torsional oscillation of the phacoemulsification tip at frequencies of about 32,000 cycles per second.

Use of the torsional-type handpiece has called for phacoemulsification needle tip designs differing from those used with the longitudinal-type handpiece. For example, needles have been designed with tips that are shaped, swaged, and angled to take advantage of the needle motion created by the handpiece.

Many surgeons favor phacoemulsification needles having the straight tip design commonly used with longitudinal handpieces. The great majority of surgeons use longitudinal handpieces rather than the torsional handpieces, often because torsional phacoemulsification equipment is more expensive than longitudinal equipment, and thus these surgeons find themselves unable to take advantage of the enhanced phacoemulsification results claimed by the torsional phacoemulsification systems.

U.S. Pat. No. 10,952,895, hereby incorporated by reference in its entirety, discloses a needle tip in an off-axis position relative to the axis of the aspiration passage extending through the needle body causes eccentric motion or “wobble” during torsional phacoemulsification and improves the efficiency of phacoemulsification while retaining the straight-tip configuration. It has also been found that forming the tip in such an off-axis position also increases the efficiency of phacoemulsification when using a longitudinal handpiece.

Use of an off-axis tip with a longitudinal hand piece appears to desirably create a hybrid type of phacoemulsification motion without using the more complex and expensive torsional phacoemulsification apparatus. The eccentric or wobble type of motion can be imparted to a phacoemulsification needle with no flare at the tip by forming the central aspiration passage within the needle body in an off-axis position. Similar results can be obtained using a straight phacoemulsification needle having an aspiration passage that is formed with a cross-sectional configuration different than the cross-sectional configuration of the needle body itself. These results will be further amplified if the passage is also placed off-axis.

The inventor has found, however, that it may be particularly difficult to cost effectively manufacture aforementioned off-axis type needles. There remains a need for phacoemulsification needles exhibiting improved performance and efficiency that can be easily manufactured in large quantities.

The present invention is directed to an improved phacoemulsification surgical instrument, having an improved needle tip configuration to promote improved emulsification efficiency, improved aspiration, the minimization of the transmission of thermal energy to the site during a procedure, and/or lowered cost and improved manufacturability of the needle.

The present invention is further directed to an improved phacoemulsification surgical instrument assembly, wherein an improved infusion sleeve cooperates with a phacoemulsification needle having a flared or enlarged tip to promote flow of irrigation fluid to the surgical site, improve sealing of the incision in the eye, reduce flutter of the cornea, while minimizing transmission of thermal energy to the site during a procedure.

The present invention is further directed to an improved phacoemulsification needle having an improved treatment on the tip, and optionally portions of the needle body, which reduces or minimizes glare under the microscope during a phacoemulsification procedure.

### SUMMARY OF THE INVENTION

The present invention is directed to an improved phacoemulsification needle which is particularly suited for use with an associated vibratory surgical handpiece, wherein the handpiece may be configured for torsional (i.e., rotational) ultrasonic movement, as well as linear or longitudinal movement, elliptical, or blended movement, etc.

In accordance with one broad form of the present invention, the phacoemulsification needle includes a needle shaft portion defining a longitudinal axis and having an internal aspiration passage. The needle includes an emulsification tip joined to a distal end of the needle shaft portion. The emulsification tip has five side walls defining a substantially pentagonal perimeter of an open mouth communicating with the aspiration passage. The open mouth defines a central, longitudinal mouth axis that is coincident with the central, longitudinal axis of the shaft portion of the needle.

In yet another preferred form of the present invention, adjacent ones of the five side walls converge at convex corners for enhanced safety during operation of the needle. Preferably, one or more of the corners defines an arc length that is less than about half of the length of one or more of the side walls. In another preferred form, one or more of the convex corners defines an arc length that is less than about one quarter of the length of one or more of the side walls.

In one preferred form of the present invention, three of the side walls define a first thickness, while the remaining two of the side walls (1) converge at a location in a vertical plane that extends through the longitudinal shaft axis, and (2) have a second thickness that is greater than the first thickness. Preferably, the first thickness is between about 0.01 mm and about 1.5 mm. Further, the second thickness is preferably between about 0.01 mm and about 1.5 mm.

According to yet another form of the present invention, the mouth of the tip portion defines a substantially circular cross-sectional shape in a plane that is perpendicular to the longitudinal shaft axis.

In another preferred form of the present invention, the emulsification tip has a distal edge disposed at an acute angle to a plane extending normally through the longitudinal axis of the needle shaft portion. Preferably, the acute angle is between about 20 and 40 degrees, and more preferably about 30 degrees.

In yet another preferred form of the present invention, two of the side walls converge in a rounded corner at a location proximate to a vertical plane that extends through the longitudinal shaft axis.

According to one preferred form of the present invention, the emulsification tip extends a length along the longitudinal shaft axis between about 1.00 mm and about 2.00 mm.

According to one broad form of the present invention, a phacoemulsification needle includes a shaft portion defining a longitudinal shaft axis, and having an internal aspiration passage. An emulsification tip is joined to a distal end of the needle shaft portion, and the emulsification tip has five side walls defining a substantially pentagonal perimeter of an open mouth communicating with the aspiration passage. The open mouth defines a longitudinal mouth axis that is offset from the longitudinal shaft axis, and the open mouth defines an oval-shaped internal surface.

According to one form of the present invention an improved phacoemulsification surgical instrument assembly, including a phacoemulsification infusion sleeve and needle, is provided for use with a phacoemulsification handpiece. The phacoemulsification needle includes a body defining a central body axis and terminates at a tip. The tip has an offset portion defining a central tip axis that is offset from the central body axis. The phacoemulsification needle defines an internal aspiration passage for aspirating an irrigating fluid from a surgical site. The infusion sleeve is positioned around at least a portion of the phacoemulsification needle such that the tip of the phacoemulsification needle extends beyond a distal end of the infusion sleeve. The infusion sleeve has a hollow, flexible tubular body with an open end through which the tip of the phacoemulsification needle protrudes, the tubular body defining an internal passage through which irrigating fluid passes to a surgical site. A portion of the distal end of the infusion sleeve engages the offset portion of the tip.

In one form of the invention, the infusion sleeve includes a pair of closely spaced, internal ribs positioned to engage the phacoemulsification needle.

According to another form of the present invention, the tip of the phacoemulsification needle has a relatively enlarged configuration compared to the body of the phacoemulsification needle.

Preferably, the tip of the phacoemulsification needle has a generally rectangular cross-section, taken in a normal plane relative to the tip central axis.

In yet another form of the present invention, the infusion sleeve includes first and second pairs of the closely spaced, internal ribs, positioned in a diametrically opposed relationship with respect to the phacoemulsification needle. The infusion sleeve preferably further includes first and second, single internal ribs positioned in diametrically opposed relationship to each other, each of the single internal ribs being positioned intermediate of the first and second pairs of internal ribs at substantially 90-degree spacing from each of the first and second pairs of internal ribs.

According to another aspect of the present invention, the distal end of the infusion sleeve engages the tip of the needle in to form a circular ring or sealing/contact surface to enhance sealing engagement between the infusion sleeve and the tip of the phacoemulsification needle.

In one aspect of the present invention, the infusion sleeve defines a pair of fluid ports communicating with the internal passage defined by the tubular body of the sleeve and the offset portion of the tip is positioned between the pair of fluid ports in the assembly. In a preferred form of the present invention, the pair of fluid ports are positioned in diametrically opposed relationship to each other, with the offset portion of the tip located centrally between the pair of fluid ports.

In still another form of the present invention, the distal end of the infusion sleeve tapers inwardly toward the tip of the phacoemulsification needle.

In one broad form of the present invention, an infusion sleeve is provided for use with a phacoemulsification handpiece having a phacoemulsification needle extending therefrom. The infusion sleeve includes a hollow, flexible tubular body through which the phacoemulsification needle extends, with an open, distal end through which a tip of the needle protrudes. The tubular body defines an internal passage through which irrigating fluid passes to a surgical site when the infusion sleeve is mounted to the handpiece. The infusion sleeve has at least one pair of closely spaced, internal ribs positioned to engage the phacoemulsification needle.

In one form of the present invention, the distal end of the phacoemulsification needle has a relatively enlarged configuration for sealingly engaging a distal end of the tubular body of the infusion sleeve at its open end.

According to yet another form of the present invention, the infusion sleeve includes first and second pairs of the closely spaced, internal ribs, positioned in diametrically opposed relationship with respect to the phacoemulsification needle.

In one preferred form of the present invention, the infusion sleeve includes first and second, single internal ribs positioned in diametrically opposed relationship to each other, each of the single internal ribs being positioned intermediate of the first and second pairs of internal ribs at substantially 90-degree spacing from each of the first and second pairs of internal ribs.

In another broad form of the present invention, the infusion sleeve defines a circular ring positioned at its distal end to enhance sealing engagement between the infusion sleeve and the relatively enlarged tip of the phacoemulsification needle.

In another form of the present invention, an infusion sleeve is provided for use with a phacoemulsification handpiece having a phacoemulsification needle extending therefrom. The sleeve includes a hollow flexible tubular body through which the phacoemulsification needle extends, and further has distal end through which a tip of the phacoemulsification needle protrudes. The tubular body defines an internal passage through which irrigating liquid passes to a surgical site when the infusion sleeve is mounted to a handpiece. The infusion sleeve defines a circular ring positioned at the distal end of the tubular body to enhance sealing engagement between the infusion sleeve and the tip of the phacoemulsification needle.

In still another broad form of the present invention, a phacoemulsification needle includes a needle shaft portion having a proximal end for being attached to a vibratory handpiece and a opposite distal end, an internal aspiration passage extending between the proximal end and the distal end, and defining a longitudinal shaft axis centered on the internal aspiration passage between the proximal end and the distal end. The needle includes a flaring, emulsification tip joined to the distal end of the needle shaft portion, and extends radially outwardly beyond the needle shaft portion, relative to the axis. The emulsification tip has an anti-glare surface treatment.

In one preferred form of the present invention, the anti-glare surface treatment extends a length along the longitudinal shaft axis across the entirety of the emulsification tip and at least a part of the needle shaft portion.

In another preferred form of the present invention, the anti-glare surface treatment has a surface finish of between N1 and N10, and more preferably a surface finish of N8.

In another broad form of the present invention, a phacoemulsification instrument includes a phacoemulsification needle. The phacoemulsification needle has a needle shaft portion having a proximal end for being attached to a vibratory handpiece and a distal end, an internal aspiration passage extending between the proximal end and the distal end, and defining a longitudinal shaft axis centered on the internal aspiration passage between the proximal end and the distal end. The needle includes a flaring, emulsification tip joined to the distal end of the needle shaft portion, the flaring emulsification tip extending radially outwardly beyond the needle shaft portion. The instrument further includes an infusion sleeve assembled around the phacoemulsification needle. The infusion sleeve has a hollow flexible tubular body receiving the phacoemulsification needle and has open proximal and distal ends. The hollow tubular body defines at least one fluid port and an internal passage through which irrigating fluid passes to a surgical site. The distal end of the infusion sleeve contacts the flaring, emulsification tip to restrict flow of an irrigating fluid from the distal end of said infusion sleeve.

In one preferred form of the present invention, the infusion sleeve includes at least one pair of adjacent, internal ribs within the internal passage positioned to engage the phacoemulsification needle.

In another preferred form of the present invention, the distal end of the infusion sleeve includes a circular ring for sealing engagement against a portion of the flaring, emulsification tip.

In still another preferred form of the present invention, at least a portion of the emulsification tip includes an anti-glare surface treatment, which more preferably extends along the entirety of the emulsification tip to a portion of the needle body.

Other features and advantages of the present invention become readily apparent from the following detailed description, the accompanying drawings, and the appended claims.

### BRIEF DESCRIPTION OF THE DRAWINGS

In the accompanying drawings forming part of the specification, in which like numerals are employed to designate like parts throughout the same,

FIG. 1 is an isometric view, from the front and right side, of a first illustrated embodiment of a phacoemulsification surgical needle of the present invention shown assembled with a vibratory handpiece shown diagrammatically;

FIG. 2 is a fragmentary, left-side elevational view of the tip portion of the instrument shown in FIG. 1;

FIG. 3 is a fragmentary, left-side elevational view of the tip portion of the instrument shown in FIG. 1, and FIG. 3 shows internal surface features in hidden/dashed lines;

FIG. 4 is a fragmentary, front elevational view of the operative tip portion of the surgical instrument shown in FIG. 1;

FIG. 5 is a fragmentary, front elevational view of the operative tip portion of a second embodiment of an instrument according to the present invention;

FIG. 6 is a fragmentary, left-side elevational view of the tip portion of the instrument shown in FIG. 5;

FIG. 7 is a fragmentary, left-side elevational view of the tip portion of the instrument shown in FIG. 5, and FIG. 7 shows internal surface features in hidden/dashed lines;

FIG. 8 is a cross-sectional view of the operative tip portion of a third embodiment of an instrument according to the present invention, taken along a vertical plane extending through a central longitudinal axis of the body and tip of the instrument;

FIG. 9 is an isometric view, from the front, top, and left side, of a fourth illustrated embodiment of a phacoemulsification surgical needle of the present invention;

FIG. 10 is a right-side elevational view of the instrument shown in FIG. 9;

FIG. 11 is a bottom plan view of the instrument shown in FIG. 9;

FIG. 12 is a front elevation view of the instrument shown in FIG. 9;

FIG. 13 is a fragmentary, cross-sectional view of the tip portion of the instrument shown in FIG. 9, taken along view line 13-13 in FIG. 11;

FIG. 14 is a greatly enlarged, fragmentary front elevational view of the tip portion of the instrument of FIG. 9;

FIG. 15 is a greatly enlarged, fragmentary, isometric view, from the front, top, and right side, of a fifth illustrated embodiment of a phacoemulsification surgical needle of the present invention;

FIG. 16 is a right-side elevational view of the instrument shown in FIG. 15;

FIG. 17 is a greatly enlarged, fragmentary front elevational view of the tip portion of the instrument of FIG. 15;

FIG. 18 is a fragmentary, diagrammatic view of a present phacoemulsification surgical instrument assembly of the present invention;

FIG. 19 is a diagrammatic cross-sectional view of the instrument assembly shown in FIG. 18;

FIG. 20 is a perspective view of only the phacoemulsification infusion sleeve of the surgical instrument assembly of the present invention shown in FIG. 18;

FIG. 21 is a diagrammatic, cross-sectional view of a prior art infusion sleeve illustrating the harmful transmission of thermal energy to a surgical site; and

FIG. 22 is a diagrammatic, cross-sectional view illustrating the minimization of transmission of thermal energy to a surgical site, in accordance with the present invention.

### DETAILED DESCRIPTION OF THE PREFERRED EMBODIMENTS

While the present invention is susceptible of embodiment in various forms, there are shown in the drawings and will hereinafter be described the presently preferred embodiments, with the understanding that the present disclosure should be considered as an exemplification of the invention, and is not intended to limit the broadest forms of the invention to only the specific embodiments illustrated.

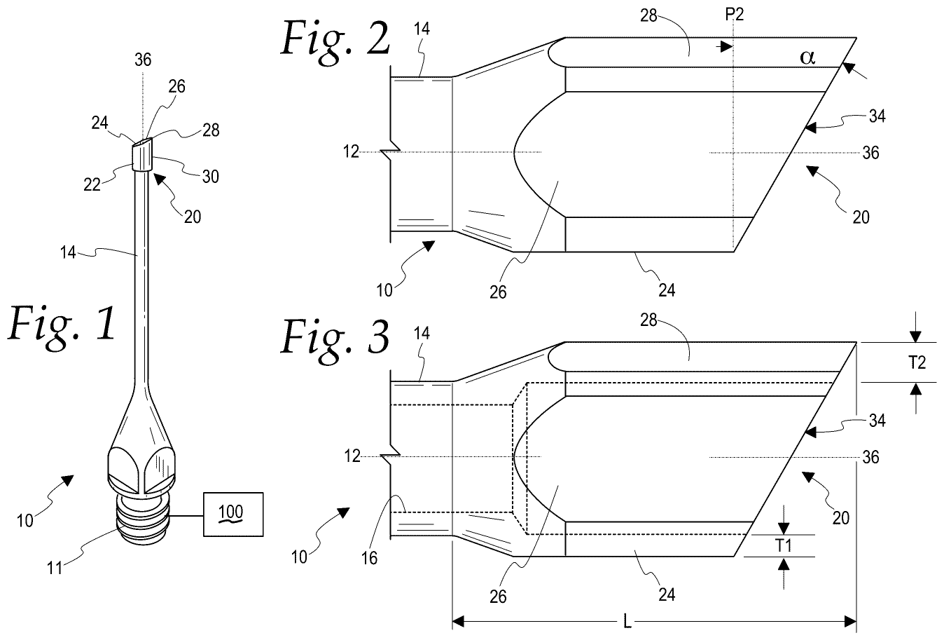

A first illustrated embodiment of a surgical instrument or phacoemulsification needle 10 according to the present invention is shown in FIGS. 1-4, wherein the needle 10 includes an elongate needle shaft portion 14 having a threaded proximal end 11 (FIG. 1) for being connected to mating threads in a vibratory handpiece 100 (shown diagrammatically in FIG. 1 only) and defining a central, longitudinal shaft axis 12 (FIGS. 2-4). The handpiece 100 may be any one of a longitudinally-vibrating handpiece, a torsionally-vibrating handpiece, an elliptically-vibrating handpiece, and/or a blend thereof. Other commercially available handpieces are contemplated. The shaft portion 14 defines an internal aspiration passage 16 (FIG. 3), through which aspiration is effected during phacoemulsification. Only a distal portion of the needle 10 is illustrated in many of the accompanying figures, and it will be understood that the proximal end 11 of the needles disclosed herein may have a variety of means or structures suited for being attached to a vibratory hand piece, such as by mating screw threads, clamps, locks, friction fitting, etc. Suitable proximal end structures of needles are described in U.S. Pat. No. 8,764,782, the entirety of which is incorporated herein by reference.

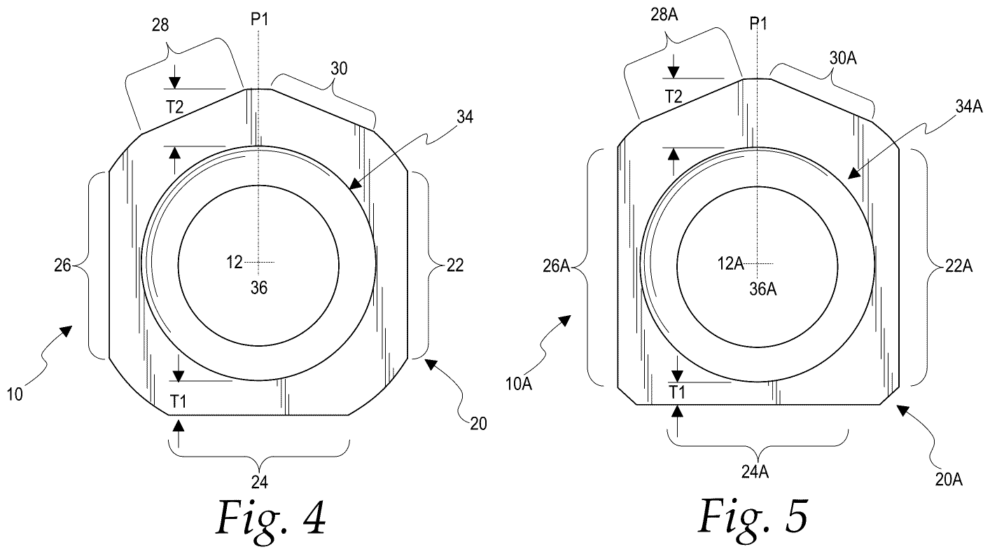

With reference to FIGS. 1 and 4, the present phacoemulsification needle 10 further comprises an operative, enlarged emulsification tip 20 joined to a distal end of the needle shaft portion 14, opposite of the proximal end 11. Notably, the emulsification tip 20 has a generally, pentagonal cross-sectional configuration in a plane extending perpendicular to the longitudinal shaft axis 12 (as shown in FIG. 4), and includes five substantially planar side walls, or simply referred to hereinafter simply as “walls”, 22, 24, 26, 28, 30 defining a perimeter of an open mouth 34. The open mouth 34, which is preferably defines an internal surface that is circular in cross-section (as viewed in FIG. 4) for ease of manufacture of the tip 20 communicates with, or connects to, the aspiration passage 16 of the needle shaft portion 14 to facilitate aspiration of emulsified tissues through the needle 10 when subjected to a vacuum.

As can be seen in FIG. 4, the first illustrated embodiment of the emulsification needle 10 desirably includes a first thickness T1 of at least one of the walls 22, 24, 26, and a substantially greater second thickness T2 of at least one of the other converging walls 28, 30, which effect phacoemulsification attendant to torsional, longitudinal, elliptical, and/or blended ultrasonic movement of the needle 10. Preferably, each of the walls 28, 30 converge at a location proximate to a vertical plane “P1” that extends through the longitudinal shaft axis 12 and have a symmetry about this vertical plane “P1”. More preferably, this plane “P1” extends through the bottom of the needle tip portion 20. Preferably, each of the walls 22, 24, 26 has the same nominal thickness T1 at its geometric center of between about 0.01 mm and about 1.5 mm, and each of the converging lower or bottom walls 28, 30 has the same nominal thickness T2 at its geometric center of between about 0.1 mm and about 1.5 mm. In the illustrated embodiment of FIG. 4, the second thickness T2 is at least two times greater than the first thickness T1 to impart the desired wobble effect during operation of the needle 10.

With reference now to FIG. 4, in the illustrated first embodiment of the instrument 10, adjacent ones of the walls 22, 24, 26, 28, 30 of the tip portion 20, defining the mouth 34, advantageously connect in rounded, convexly-curved corners for enhanced safety in the eye. One or more of the convex corners between the side walls 22, 24, 26, 28, 30 defines an arc length that is about half of the length of the adjacent side walls for enhanced safety during operation.

With reference to FIG. 3, the emulsification tip 20 preferably extends from the connection to the shaft 14 a length “L” along the longitudinal shaft axis 12 between about 1.00 mm and about 2.00 mm.

With reference to FIG. 2, the emulsification tip 20 has a distal edge disposed at an acute angle α of between about 20 and 40 degrees, more preferably about 30 degrees, relative to the plane “P2” extending normal or perpendicular to the longitudinal axis 12 of the needle shaft portion 14. This arrangement facilitates efficient use, and is particularly suitable for use with an ultrasonic instrument configured to move ultrasonically in linear and/or torsional modes.

With reference to FIG. 3, the mouth 34 defines a central, longitudinal mouth axis 36 that is substantially coincident with the longitudinal axis 12 of the needle shaft portion 14, to enhance manufacturability of the needle 10 while simultaneously providing an advantageous emulsifying or chopping action of the thickened converging, bottom walls 28, 30 during vibratory operation of the needle 10. The coaxial alignment of the axes 12 and 36 permit a single milling, drilling, or other material removal process to be used to significantly reduce manufacturing complexity and costs compared to prior art needle configurations.

A desirable feature of the present phacoemulsification needle is the absence of sharp edges on the exterior of the needle 10. The forward, distal edges of the emulsification tip 20 are preferably rounded and smooth, without sharp edges. There are preferably no sharp edges on the outer periphery of the tip 20.

The specific configuration of the present needle 10 can be varied depending upon intended use. The needle shaft portion 14 can be straight as in the illustrated embodiments, or the needle shaft portion 14 may be bent to a Kelman-style configuration for effecting chopping during a surgical procedure. Alternatively, the needle shaft portion 14 can be bent to an Akahoshi-style (Reverse Kelman Bend) for pre-chopping during a procedure. Other bent configurations are contemplated.

The outer surface of the tip 20 may have a sandblasted finish to eliminate or at least reduce the potential for sharp edges for improved safety of the instrument 10 in the eye. Such sandblasting or other conventional or non-conventional finishing methods may be applied to the needle embodiments discussed below.

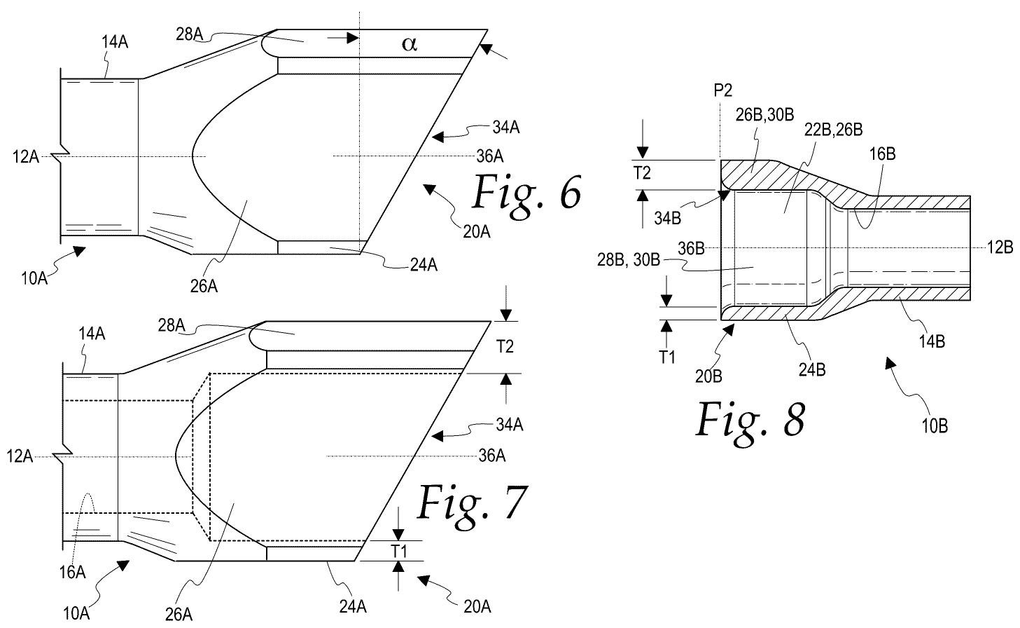

A second embodiment of a surgical needle or instrument according to the present invention is shown in FIGS. 5-7, designated by the numeral 10A, and functions similarly to the first illustrated embodiment of the needle 10 as previously described in FIGS. 1-4. The numbered features of the second embodiment of the needle 10A illustrated in FIGS. 1-4 are analogous to features of the first embodiment of the needle 10 that share the same number, but without the suffix “A”. The second embodiment of the surgical needle 10A includes an emulsification tip 20A with a generally, pentagonal cross-sectional configuration, and includes a five substantially planar side walls (when viewed on the exterior of the needle tip 20A), or simply “walls”, 22A, 24A, 26A, 28A, 30A defining a perimeter of an open mouth 34A, which has a circular interior surface or cross-sectional shape.

As can be seen in FIG. 5, the walls 22A, 24A, 26A, 28A, 30A connect to one another in significantly shorter rounded, convexly-curved corners as compared to the first embodiment of the needle 10 described above. The convex corners can be seen to define an arc length that is much less than a half, preferably less than a third or a quarter of the length of the adjacent side walls. In addition, the tip 20A differs further from the prior embodiment of the needle 10 in that the first thickness T1 is significantly reduced compared to the second thickness T2 of the converging walls 28A, 30A, wherein the second thickness T2 is at least three times the thickness of the first thickness T1 of the opposite wall 24A.

The second illustrated embodiment of the needle 10A may provide a more dramatic wobble effect during phacoemulsification as compared to the first illustrated embodiment of the needle when coupled with some vibratory handpieces.

A third embodiment of a surgical needle according to the present invention is shown in FIG. 8, designated by the numeral 10B, and functions similarly to the first illustrated embodiment of the needle 10 as previously described in FIGS. 1-4. The numbered features of the third embodiment of the needle illustrated in FIG. 8 are analogous to features of the first embodiment of the needle that share the same number (without the suffix “B”). The third embodiment of the surgical needle 10B differs from the aforementioned first illustrated embodiment in that the needle 10B includes an emulsification tip 20B with a flat termination of the pentagon-shaped walls 22B, 24B, 26B, 28B, 30B, wherein the walls terminate generally within a shared plane P2 that is perpendicular to the coincident axes 12B and 36B. Furthermore, the lead-in surfaces of the mouth 34B are rounded for enhanced safety to the lens capsule.

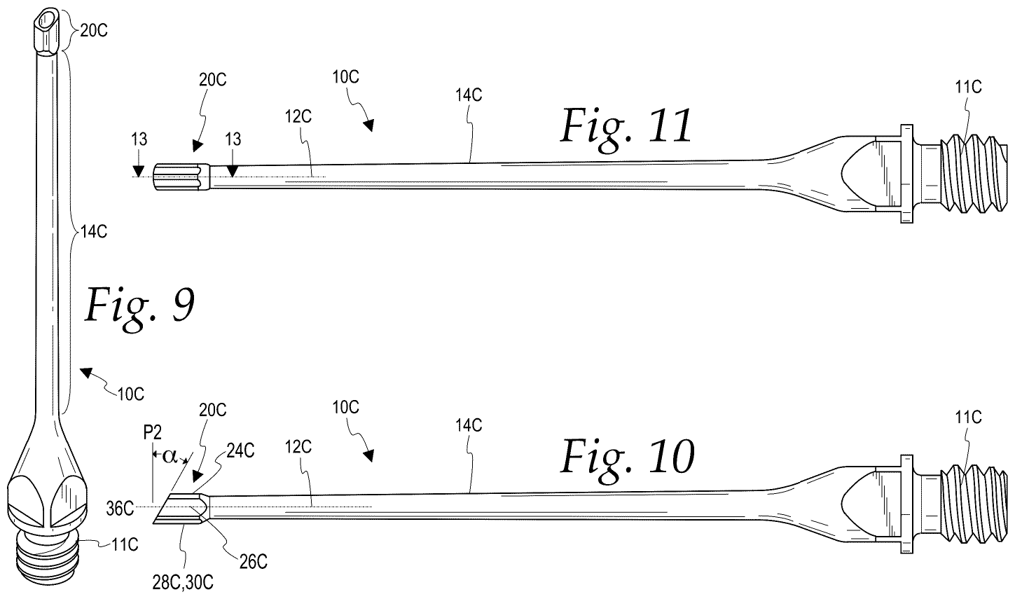

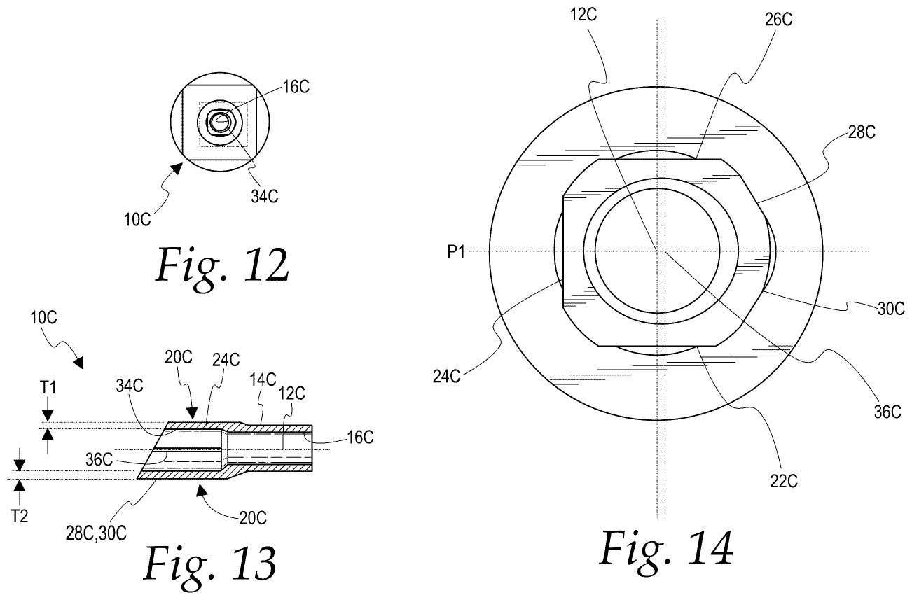

A fourth embodiment of a surgical needle or instrument according to the present invention is shown in FIGS. 9-14, designated by the numeral 10C, and functions similarly to the first illustrated embodiment of the needle 10 as previously described in FIGS. 1-4. The numbered features of the fourth embodiment of the needle 10C illustrated in FIGS. 9-14 are analogous to features of the first embodiment of the needle 10 that share the same number, but without the suffix “C”. The fourth embodiment of the surgical needle 10C includes an emulsification tip 20C with a generally, pentagonal cross-sectional configuration, and includes a five substantially planar side walls (when viewed on the exterior of the needle tip 20C), or simply “walls”, 22C, 24C, 26C, 28C, 30C defining a perimeter of an open mouth 34C, which, unlike the prior-discussed embodiments, has an oval interior surface or cross-sectional shape. The inventor has found that the oval shaped opening provides an increased aspiration rate at the tip 20C of the needle 10C, compared to the other embodiments disclosed herein.

With reference to FIG. 14, the mouth 34C defines a central, longitudinal mouth axis 36C that is substantially parallel to, and offset from, the longitudinal axis 12C of the needle shaft portion 14C, to provide an advantageous additional emulsifying or chopping action of the thickened converging, bottom walls 28C, 30C during vibratory operation (longitudinal, torsional, or blended vibratory action) of the needle 10C. It is believed that the weight at the top and bottom of the pentagon shaped tip is unequal. With reference to FIG. 13, the narrowing of the mouth 34C includes dual emulsification points inside of the tip 20C (at the locations transitioning to the aspiration passage 16C of the needle body 14C to assist in secondary emulsification of the nucleus.

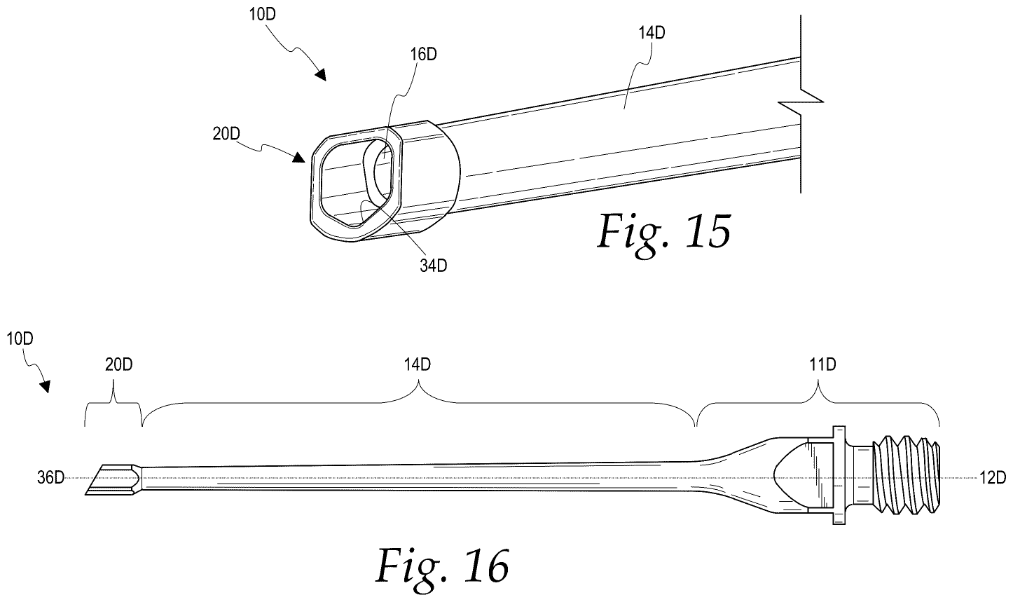

A fifth embodiment of a surgical needle or instrument according to the present invention is shown in FIGS. 15-17, designated by the numeral 10D, and functions similarly to the first illustrated embodiment of the needle 10 as previously described in FIGS. 1-4. The numbered features of the fifth embodiment of the needle 10D illustrated in FIGS. 15-17 are analogous to features of the first embodiment of the needle 10 that share the same number, but without the suffix “D”. The fifth embodiment of the surgical needle 10D includes an emulsification tip 20D with a generally, pentagonal cross-sectional configuration, and includes a five substantially planar side walls (when viewed on the exterior of the needle tip 20D), or simply “walls”, 22D, 24D, 26D, 28D, 30D defining a perimeter of an open mouth 34D, which, unlike the prior-discussed embodiments, has pentagonal interior surface or cross-sectional shape. The inventor has found that the pentagonal shaped opening provides an unequal weight distribution at the top and bottom of the pentagonal tip portion 20D, improved aspiration, flow and vibration characteristics compared to needles of the prior art. The needle 10D performs advantageously in longitudinal, torsional, or hybrid type vibratory modes.

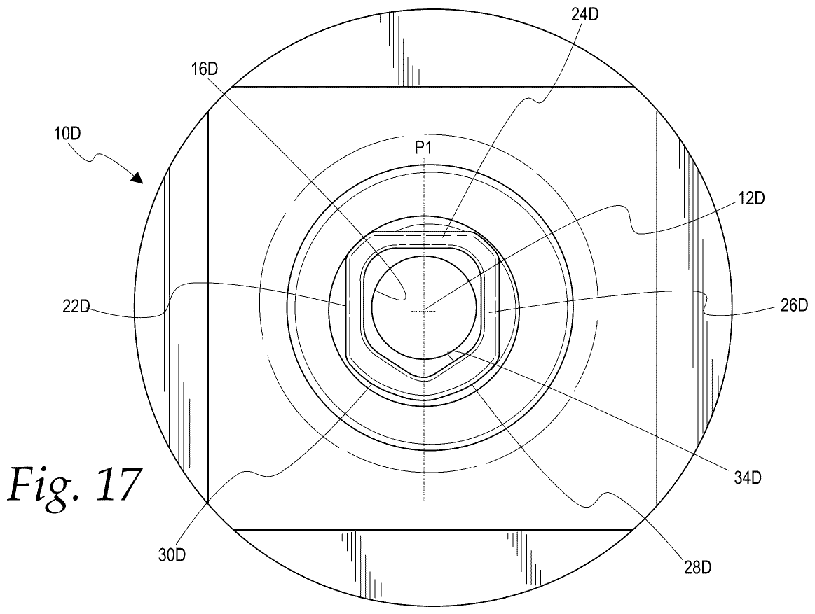

With reference to FIG. 17, the geometric center longitudinal axis 36D of the mouth 34D is coaxially aligned with the central, longitudinal axis 12D of the needle shaft portion 14D. The walls 22D, 24D, 26D, 28D, 30D connect to one another in rounded, convexly-curved corners.

The inventor believes that the needle designs described herein exhibit better followability of the nucleus, better fluidics, no observable chatter, repulsion, or bubbles regardless of nucleus grade, no milk-like fluid when emulsifying a grade 4 or 5 nucleus, improved sculpting or grooving of the nucleus, improved chamber stability, and better overall efficiency when compared to prior art phacoemulsification needles. It is currently believed that the pentagonal or pentagonal-like shape of the needle tip portions described above creates a vortices to increase the volume of inflow of balanced salt solution and emulsified nucleus in the tip has increased followability. The inventor has further found that the needle 10C exhibits overall improvements of flow and vibration characteristics compared to the offset needles of the prior art. In one study, the velocity of aspirated fluent material at the tip 20C was found to be doubled, and volume of aspirated fluent material to be increased by 50-60%, compared to an offset needle of the prior art.

The present invention further contemplates an improved phacoemulsification surgical instrument assembly, including a phacoemulsification infusion sleeve and phacoemulsification needle, which cooperate to facilitate efficient use by the surgeon, reduce the leakage of fluid through an incision in the eye, reduce flutter, and limit or reduce excessive heating of the surgical site during a phacoemulsification procedure.

The inventor of the present invention has further found that the above-discussed needles may exhibit increased vacuum output for a given setting on a vacuum source connected to the handpiece when compared to conventional needles. The inventor believes that the flared tips of the present invention create a magnetic-like followability of the nucleus during use, which improves the aspiration through the needle. The tips further exhibit multiple points of emulsification of the lens (located at the distal most end of the tip and further located within the sloping wall joining the aspiration passage with the tip to reduce clogging, minimize repulsion of the aspirated lens, and minimize anterior chamber instability.

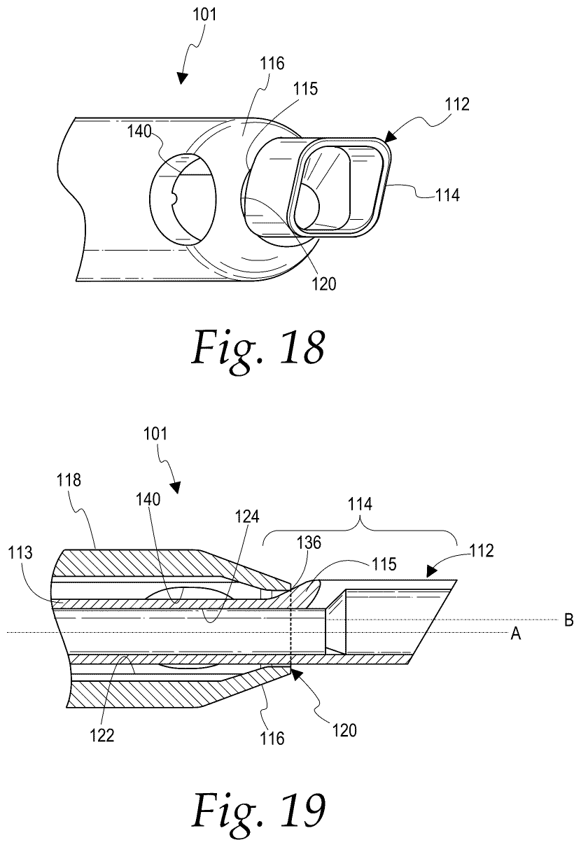

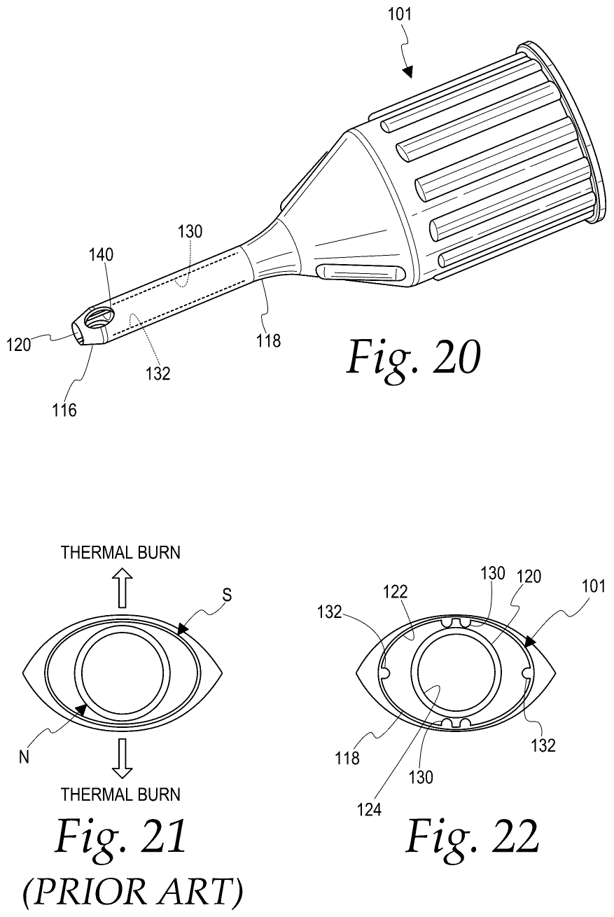

As will be further described, the infusion sleeve includes a circular ring at an open or distal end thereof for enhancing sealing engagement with a portion of the phacoemulsification needle. In the illustrated embodiment of the sleeve 101 in FIGS. 18-20 and 22, the phacoemulsification needle of the assembly comprises a relatively enlarged or flaring, offset tip, preferably having an asymmetrical configuration, with the circular ring enhancing sealing engagement between the infusion sleeve 101 and the relatively enlarged tip of the phacoemulsification needle 112.

Notably, the infusion sleeve 101 includes at least one pair of closely spaced internal ribs for engagement with the phacoemulsification needle 112, which act to desirably inhibit thermal burn attendant to ultrasonic operation of the phacoemulsification needle 112.

In accordance with the illustrated embodiment, the present phacoemulsification surgical instrument assembly includes the phacoemulsification needle 112 for being secured to, and extending from, an associated handpiece (not shown), which ultrasonically drives or vibrates the needle. The assembly further includes the infusion sleeve 101 positioned about the phacoemulsification needle 120, with a distal, operative tip 114 of the phacoemulsification needle 112 extending beyond an open, distal end 116 of the infusion sleeve 101.

With reference to FIG. 19, the phacoemulsification infusion sleeve 101 has a hollow flexible tubular body 118, with an opening 120 located at the distal end 116 through which the tip 114 of needle 112 protrudes. The tubular body 118 defines an internal passage 122 through which irrigating liquid passes, to allow irrigating liquid to flow to the capsular bag through an annular channel formed between needle 112 and sleeve 101. Irrigating fluid is supplied to the sleeve 101 when the sleeve is mounted to the handpiece. In turn, the phacoemulsification needle 112 is hollow and includes a substantially uniform, cylindrical body portion 113 defining a central body axis “A” defining an internal aspiration passage 124 for aspirating the irrigating liquid and emulsified particles of a lens from the capsular bag. In the preferred form of the invention, the tip 114 of the phacoemulsification needle 112 has a relatively enlarged or offset configuration defining a central tip axis “B” for sealingly engaging the distal end 116 of the tubular body 118 of the phacoemulsification sleeve 101 at the open end 120 thereof.

With reference to FIG. 22, the infusion sleeve 101 includes a first and second pair of closely spaced, internal ribs 130 positioned to engage the exterior surface of the phacoemulsification needle 112. The first and second pairs of closely spaced, internal ribs 130 are preferably semi-circular in cross-sectional shape and extend along the central longitudinal axis of the sleeve 101 from the tapered, distal end 116 of the sleeve 101 to a proximal end of the sleeve 101.

In one presently preferred embodiment of the invention, the phacoemulsification needle 112 is configured with a generally square cross-section defined by four adjacent walls, when viewed in a plane that is normal to the central tip axis “B”. The tip 114 of the needle 112 defines an offset portion 115 having a sloping or angled external surface.

Thus, the infusion sleeve 101 cooperates with the sloping or angled external surface of the offset portion 115 of the tip 114 of the phacoemulsification needle 112 to fit snugly, creating a plug-like effect, thereby preventing leakage of irrigating fluid, and also desirably providing a snug fit with the incision at the surgical site. The engagement of the infusion sleeve 101 with the offset portion of the tip 114 may be said to define a generally circular seal or circular ring 136, positioned at the open end 120 of the tubular body 118 of the infusion sleeve 101, to enhance sealing engagement between the sleeve 101 and the relatively enlarged, tip 114 of the phacoemulsification needle 112.

It should be noted that a needle 112 configured as described above can be readily inserted in a relatively small incision, such as 2.0 mm, by insertion of the side or lateral region to the tip 114, rather a straight entry insertion. Moreover, the shape of the distal tip 114 of needle 112 should not be confused with the bevel of conventional phacoemulsification needles since the contemplated tip 114 does not have any bevel. When the tip 114 is angled downwardly with respect to the axis “A” of the needle body 113, in the so-called “Akahoshi bend”, it has been found by the inventor to allow better grasp of the nuclear fragments without breaking occlusion.

Referring to FIG. 22, in the illustrated embodiment, the phacoemulsification infusion sleeve 101 includes first and second pairs of the closely spaced, internal ribs 130, positioned in diametrically opposed relationship with respect to each other. In the preferred form, the infusion sleeve 101 further includes first and second, single internal ribs 132 positioned in diametrically opposed relationship to each other, with each of the single internal ribs 132 being positioned intermediate of the first and second pairs of internal ribs 130, at substantially 90-degree spacing from each of the first and second pairs of internal ribs 130.

The phacoemulsification infusion sleeve 101 preferably defines a pair of fluid ports 140 communicating with the internal passage 122 defined by the tubular body 118 of the sleeve 101. The offset portion 115 of the tip 114 of the phacoemulsification needle 112 is preferably positioned between the pair of fluid ports 140. More preferably, the pair of fluid ports 140 are positioned in diametrically opposed relationship to each other, with the offset portion 115 of the tip 114 of the phacoemulsification needle 112 positioned centrally between the pair of fluid ports 140. This desirably acts to form a V-shape when irrigation is started.

With reference now to FIGS. 21 and 22, the advantages provided by the present invention are diagrammatically illustrated.

The sleeve of the present invention, shown diagrammatically in FIG. 22, is different than a conventional sleeve, as shown in FIG. 21. As shown in FIG. 21, the conventional infusion sleeve “S” shown surrounding an associated phacoemulsification needle “N” can cause thermal burn as thermal energy is transferred from the ultrasonically actuated needle to tissue at the surrounding “fish mouth” incision. In significant contrast, the assembly of the present invention, including the pairs of closely positioned, internal ribs 130, desirably reduces and abates such thermal transfer.

The features of the present invention, including the offset portion 115 of the tip 114 of the phacoemulsification needle 112, and engagement of the distal, collar or ring feature of the infusion sleeve 101, desirably act to plug the so-called “fish mouth” effect on the incision which is created when a phacoemulsification needle tip 114 and sleeve 101 are introduced into the linear cataract incision. The linear cataract incision resembles an opening looking like a fish mouth, and it is a main object to maintain fluid inside the eye/anterior chamber. Regardless of the incision size, the fish mouth opening is based on the dimension of the phacoemulsification needle tip 114 and infusion sleeve 101.

It has been surprisingly noted by the inventor that the same tip 114 and sleeve 101 fit for different incision sizes. For example, the tip 114 can be 0.9 mm in length (along one of the sides of the square tip), while the sleeve 101 with the pairs of internal ridges 130 shown in FIG. 22 is different from the conventional sleeve S as shown is FIG. 21. This allows the sleeve 101 to be flexible and snug when the incision size is small, and the sleeve 101 does not collapse when the pressure is high. Hence, there is no appreciable thermal burn seen at the incision spot. Even through a 2.0 mm incision, the same tip 114 can be inserted. When the incision size is larger (2.2 mm, 2.4 mm, 2.6 mm) the sleeve 101 is not compressed as much and hence retains the full shape and is able to perform well without leakage.

In the illustrated embodiment, the tubular body 118 of the infusion sleeve 101 is shown as having a generally circular cross-section, such that the anterior (top) and posterior (bottom) surfaces are generally the same. However, these surfaces can be significantly different. The anterior surface has been provided with outer ridges and can have various textures to remove epithelial cells from the anterior capsule, and to polish the anterior capsule, with a convex configuration. The posterior surface can have ridges to remove cells to prevent migration to an intraocular lens with a concave configuration.

It is contemplated that other variations in port size, number and positioning may also be used. As a general rule, the stiffer the material used to form the sleeve 101, the more ports 140 may be used. Stiffer material will keep the sleeve 101 from collapsing during surgery and touching the needle 112 or otherwise affecting the rate of flow of infusing liquid through the sleeve 101.

It is further contemplated that the sleeve 101 may be used with other needle configurations, such as the other needles 10, 10A, 10B, 10C, 10D discussed above, or other conventional or non-conventional needles.

The tip portions of the needles 10, 10A, 10B, 10C, 10D, 112, discussed above, or other conventional or non-conventional needles, may further be provided with an anti-glare surface treatment of the present invention. The inventor has found that the anti-glare surface treatment applied to the tip portions of the above-discussed needles, may greatly improve the visibility of the needles during a phacoemulsification procedure within the eye. The treatment is particularly beneficial under a microscope used by the surgeon. The treatment may further remove sharp edges of the leading portion or edge of the tip to protect the posterior capsule from rupture or damage during use. The matte treatment may further improve emulsification of the lens by minimizing the adherence or stickiness of the lens to the surfaces of the tip and needle body. Preferably, the anti-glare surface treatment extends a length along the longitudinal shaft axis of the needle across the entirety of the emulsification tip. It is further preferable that the treatment extends to at least a part of the outer surface of the needle shaft portion. Preferably, the treatment extends a length along the axis of longitudinal shaft axis of between about 2 mm and 4 mm, and more preferably about 2.3-3.8 mm. The surface treatment may be defined by an N1-N10 surface finish, and preferably an N8 surface finish has been found to exhibit improved visibility and glare reduction (i.e., decreased light scattering).

In one preferred method, the treatment is formed by using 50 micron crushed glass via sandblasting. Other methods may be feasible, such as chemical etching.

From the foregoing, it will be observed that numerous modifications and variations can be effected without departing from the true spirit and scope of the novel concept of the present invention. It is to be understood that no limitation of the broadest concepts with respect to the specific embodiments illustrated herein is intended or should be inferred. The disclosure is intended to cover, by the appended claims, all such modifications as fall within the scope of the claims.

1. A phacoemulsification needle, comprising:
a needle shaft portion having a proximal end for being attached to a vibratory handpiece and a distal end, an internal aspiration passage extending between said proximal end and said distal end, and defining a longitudinal shaft axis centered on said internal aspiration passage between said proximal end and said distal end; and
a flaring, emulsification tip joined to said distal end of said needle shaft portion, said flaring emulsification tip extending radially outwardly beyond said needle shaft portion; and at least a portion of said emulsification tip having an anti-glare surface treatment.

2. The phacoemulsification needle in accordance with claim 1, wherein said anti-glare surface treatment extends a length along said longitudinal shaft axis across the entirety of the said emulsification tip to at least a part of said needle shaft portion.

3. The phacoemulsification needle in accordance with claim 1, wherein said anti-glare surface treatment has a surface finish of between N1 and N10.

4. The phacoemulsification needle in accordance with claim 3, wherein said anti-glare surface treatment has an N8 surface finish.

5. The phacoemulsification needle in accordance with claim 1, wherein said emulsification tip has five side walls defining a substantially pentagonal perimeter of an open mouth communicating with said aspiration passage, wherein said open mouth defines a longitudinal mouth axis that is coincident with said longitudinal shaft axis.

6. The phacoemulsification needle in accordance with claim 1, wherein said emulsification tip has five side walls defining a substantially pentagonal perimeter of an open mouth communicating with said aspiration passage, wherein said open mouth defines a longitudinal mouth axis that is offset from said longitudinal shaft axis.

7. The phacoemulsification needle in accordance with claim 6, wherein said open mouth defines an oval-shaped internal surface.

8. The phacoemulsification needle in accordance with claim 1, wherein said emulsification tip has four side walls defining a substantially square perimeter of an open mouth communicating with said aspiration passage, wherein said open mouth defines a longitudinal mouth axis that is offset from said longitudinal shaft axis.

9. The phacoemulsification needle in accordance with claim 1, wherein said emulsification tip has four side walls defining a substantially square perimeter of an open mouth communicating with said aspiration passage, wherein said open mouth defines a longitudinal mouth axis that is coincident with said longitudinal shaft axis.

10. The phacoemulsification needle in accordance with claim 1 in combination with a vibratory handpiece.

11. The phacoemulsification needle in accordance with claim 1 in combination with an infusion sleeve, said infusion sleeve having a hollow flexible tubular body for receiving a phacoemulsification needle and having open proximal and distal ends, said hollow tubular body defining an internal passage through which irrigating fluid passes to a surgical site when said infusion sleeve is mounted to a handpiece, said infusion sleeve having at least one pair of adjacent, internal ribs within said internal passage positioned to engage said phacoemulsification needle.

12. An infusion sleeve for use with a phacoemulsification handpiece having a phacoemulsification needle extending therefrom, said infusion sleeve comprising:
a hollow flexible tubular body for receiving a phacoemulsification needle and having open proximal and distal ends, said hollow tubular body defining an internal passage through which irrigating fluid passes to a surgical site when said infusion sleeve is mounted to a handpiece,
said infusion sleeve having at least one pair of adjacent, internal ribs within said internal passage positioned to engage said phacoemulsification needle.

13. The infusion sleeve for use with a phacoemulsification handpiece in accordance with clam 12, wherein said infusion sleeve includes first and second pairs of said closely spaced, internal ribs, positioned in diametrically opposed relationship with respect to a central longitudinal axis of said hollow flexible tubular body.

14. The infusion sleeve for use with a phacoemulsification handpiece in accordance with claim 13, wherein said infusion sleeve further comprises first and second, single internal ribs positioned in diametrically opposed relationship to each other, each of said single internal ribs being positioned intermediate of said first and second pairs of internal ribs at substantially 90-degree spacing from each of said first and second pairs of internal ribs.

15. The infusion sleeve for a phacoemulsification handpiece in accordance with claim 11, wherein said infusion sleeve defines a circular ring positioned at said distal end of said tubular body for sealingly engaging a tip of a phacoemulsification needle assembled with said infusion sleeve.

16. The infusion sleeve for a phacoemulsification handpiece in accordance with claim 11, wherein said at least one pair of adjacent, internal ribs has the form of two spaced-apart semi-circular cross-sectioned ribs, when viewed in a plane that is normal relative to a central longitudinal axis of said hollow flexible tubular body.

17. The infusion sleeve for a phacoemulsification handpiece in accordance with claim 11, wherein said at least one pair of adjacent, internal ribs extends a majority of a length of said tubular body along the direction of a central longitudinal axis of said hollow flexible tubular body.

18. A phacoemulsification instrument, comprising:
a phacoemulsification needle, said phacoemulsification needle having a needle shaft portion having a proximal end for being attached to a vibratory handpiece and a distal end, an internal aspiration passage extending between said proximal end and said distal end, and defining a longitudinal shaft axis centered on said internal aspiration passage between said proximal end and said distal end; and a flaring, emulsification tip joined to said distal end of said needle shaft portion, said flaring emulsification tip extending radially outwardly beyond said needle shaft portion; and
an infusion sleeve assembled around said phacoemulsification needle, said infusion sleeve having a hollow flexible tubular body receiving said phacoemulsification needle and having open proximal and distal ends, said hollow tubular body defining at least one fluid port and an internal passage through which irrigating fluid passes to a surgical site, and wherein said distal end of said infusion sleeve contacts said flaring, emulsification tip to restrict flow of an irrigating fluid from said distal end of said infusion sleeve.

19. The phacoemulsification instrument in accordance with preceding claim 18 wherein said infusion sleeve includes at least one pair of adjacent, internal ribs within said internal passage positioned to engage said phacoemulsification needle.

20. The phacoemulsification instrument in accordance with claim 18 wherein said distal end of said infusion sleeve includes a circular ring for sealing engagement against a portion of said flaring, emulsification tip.

21. The phacoemulsification instrument in accordance with claim 18 wherein at least a portion of said emulsification tip includes an anti-glare surface treatment.
